(19) United States
(12) Patent Application Publication
(10) Pub. No.: US 2010/0041963 A1
(43) Pub. Date: Feb. 18, 2010

(54) Cerebral and Cardiovascular Disease Prevention Using Optical -Magnetic Resonance Hybrid Imaging

(75) Inventor: Sultan Haider, Erlangen (DE)

Correspondence Address: SIEMENS CORPORATION;INTELLECTUAL PROPERTY DEPARTMENT, 170 WOOD AVENUE SOUTH, ISELIN, NJ, 08830, US

(73) Assignee: Siemens Aktiengesellschaft, Munchen (DE)

(21) Appl. No.: 12/192,216

(22) Filed: Aug. 15, 2008

(51) Int. Cl. A61B 5/00

(52) U.S. Cl. 600/301; 382/128

(57) ABSTRACT

The invention provides a computer implemented method for calculating a chronic disease risk index for a patient undergoing a first examination, the method comprising providing a first result of the first examination wherein the first examination is selected from a group of medical imaging method;
providing a second result of a second examination wherein the second examination being related to the examination of the patient's eye; Processing in combination the first result and the second such as to calculated a combined result wherein the combined result relates the first and the second result; Classifying the combined result; Calculating the risk index for the patient. The risk index is based on the combined result or on the classified combined result.

### FIELD OF THE INVENTION

The invention relates to medical data processing, in particular to medical image processing.

### BACKGROUND OF THE INVENTION

Advances in the medical sciences and medical technology have blessed modern society with the gift of longevity.

The blessing however comes at a cost. The older on average the population gets the more likely it is that a considerable proportion of the population is afflicted with chronic diseases.

Statistical studies evidence an exponential rise especially in cerebral and cardiovascular diseases, both being by far the most common chronic diseases.

This rise is putting a tremendous financial pressure on the health care systems worldwide.

In order to advance the point of early detection of chronically diseases governments have put a number of programs for screening check-ups in place.

Diabetes screening programs are examples of such measures aiming at early detection of chronic diseases.

Modern medical technology has a number of methods at hand for early detection of a number of chronic diseases.

Sophisticated as they are, these methods are expensive, time-consuming and inconvenient for the patient.

Some of the most prominent prior art methods rely on highly dedicated imaging equipment called imaging “modalities”. Examples for those modalities are Magnetic Resonance Imaging (MRI), Computer Tomography (CT), Ultra Sound (US) and X-Ray Imaging.

Yet further, the diagnostic methods based on image material acquired by means of those modalities have a relatively narrow scope. In order to come to a conclusion as to the underlying disease or diseases a number of rounds of image acquisitions may be necessary. This further drives costs and aggravates side effects for the patient.

Therefore there is a need for a quick, simple and cost-effective system and method for supporting decision making and reaching of conclusions in the diagnostic process for the purposes of early detection of chronic diseases.

There is further a need for a system and a method supportive to the diagnostic process having a wide scope. The methods and systems therefore should be suitable for the early detection per session of not merely one, but a number of diseases.

There is further a need for a method and system suitable for use on the occasions of the above mentioned routine check-ups that are already in place.

There is also need for methods and systems convenient and less cumbersome for the patient.

### SUMMARY OF THE INVENTION

The invention addresses the above needs by providing a computer implemented method for calculating a chronic disease risk index for a patient undergoing a first examination, the method comprising:
Providing a first result of the first examination wherein the first examination is selected from a group of medical imaging methods;
Providing a second result of a second examination wherein the second examination is related to an examination of a patient's eye;
Processing the first result and the second result in combination such as to calculate a combined result wherein the combined result relates to both, the first and the second result;
Classifying the combined result;
Calculating the risk index for the patient. The risk index is based on the combined result or on the classified combined result.

By calculating the highly reliable chronic disease risk index according to the method of the present invention a number of otherwise necessary examination methods using dedicated and expensive imaging technologies can be rendered superfluous.

Yet, the disease index supports the medical practitioner in reaching a conclusion about the most appropriate subsequent examination method, if any, or an appropriate treatment.

By “processing in combination” to obtain a “combined result” is meant, that the results are processed together in an integrated manner, such as to incorporate and consolidate the first and the second result into a summarizing whole.

By “first examination” is meant a regularly scheduled, conventional screening programme already in place. During this first examination the “first result”, for example an MRI image, is acquired by means of an MRI, CT or X-ray imaging modality known in the art.

The invention takes advantage of the occasions of the first examination to widen a diagnostic scope of that first examination by obtaining the “second result” as a digital image of the patient's eye in the “second examination”.

The “second examination” is the acquisition of the digital image of the patient's eye. The second examination is a simple and yet effective procedure as the digital image can be used for the detection of a number of chronic diseases rather than the detection of merely one disease, thus widening the diagnostic scope. Information gained for diagnoses-supporting purposes is thus maximized.

The method according to the present invention rests on the observation that certain physiological features in the eye are highly indicative not only to one but a number of chronic diseases.

By means of a comparably simple optical detection device the physiological features are translated into digital geometrical objects. The indications of chronic diseases are reflected in geometrical relationships and/or properties between those geometrical objects.

The physiological features of the human eye found to be particularly useful for the method are the vessels in the retina, that is, arteries and veins, and the papilla.

The optical detection device can be a digital camera in communication with a conventional slit lamp, a pupillometer, an ophthalmoscope or a fluorescence angiographic device.

According to one aspect of the present in invention, the second examination can be carried out concomitantly to the first examination.

According to another aspect of the present invention, the first examination and the second examination are performed in combination or are performed separately, for example in subsequently. However the first and the second result are obtained together.

This adds flexibility to the inventive method and allows applicability in a wide range of circumstances.

According to a yet further aspect the method comprises obtaining in a first phase reference values from previous examinations.

According to another aspect of the invention the first result or the second result comprise at least:
detecting the first result and the second result of at least a previous examination;
matching those results out of the group of the first result and the second result which have the same detection times.

According to a further aspect of the present invention the processing of the first result or the processing of the second result comprise at least:
Extracting relevant features from the first result or from the second result;
Statistically processing the first result or the second result;
Selecting relevant parts of the first result or the second result or segmenting image in relevant image segments;
Accessing a database for employing rules, the rules being used to relate the first result or the second result in order to form a combined result;
Accessing a database for comparing the first result or the second result with reference values for evaluating a conclusion with respect to the chronic disease risk index for the patient.

The geometrical properties of and the relationships between the objects can be captured by processing and acquiring suitable statistical parameters using standard statistical software packages.

The method according to the present invention is essentially “hybrid” in that it uses images gained from the medical imaging modalities in combination with digital images acquired by using the camera or other optical devices suitable for examining the patient's eye.

According to one aspect of the present invention the geometrical objects have properties such as shape, texture and colour. The properties are compared to corresponding properties of reference data. The first result or the second result is compared with predefined “pattern” properties of reference objects.

According to one aspect of the present invention the method is performed automatically.

The invention furthermore addresses the above identified needs by providing a computer based system for calculating a chronic disease risk index for the patient undergoing the first examination. It comprises the medical imaging device, the optical detection device and a processing unit and interfaces between the processing unit and the optical detection device and the imaging devices and a reference database.

Furthermore the invention addresses the above needs by providing a computer readable medium having computer-readable instructions suitable for performing the method according to the present invention.

### BRIEF DESCRIPTIONS OF THE FIGURES

FIG. 1 shows a schematic block diagram of basic components of a computer implemented system for calculating the disease risk index according to the present invention

FIG. 2 shows a schematic flow chart of the method for calculating a disease risk index according to the present invention

### DETAILED DESCRIPTION OF THE INVENTION

Embodiments of a method for calculating a disease risk index are described hereinafter. In the following description, meaning of specific details is given to provide a thorough understanding of embodiments of the invention. One skilled in the relevant art will recognize, however, that the invention can be practiced without one or more of the specific details, or with other methods, modules, entities etc. In other instances, well-known structures, computer related functions or operations are not shown or described in detail, as they will be understood by those skilled in the art.

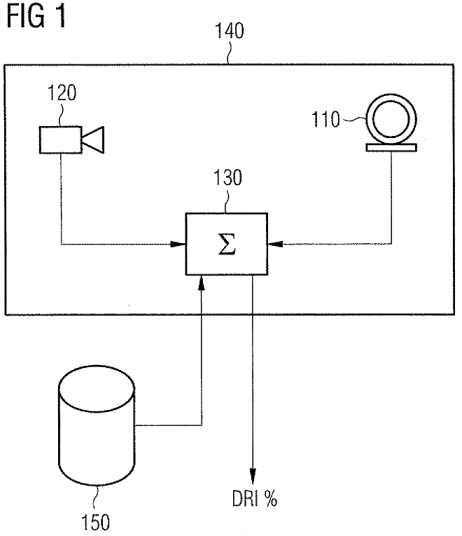

FIG. 1 shows the basic components of a computer based system for calculation of a disease risk index DRI according to the present invention.

The computer based system comprises a hybrid imaging device 140.

The hybrid imaging device 140 in turn comprises the optical detection device 120 and the medical imaging device 110. The optical detection device 120 and the medical image device 110 are arranged to communicate via a communication network with a processing unit 130.

The communication network (not shown) can be for example based on the TCP/IP (Transmission Control Protocol/Internet Protocol) protocol suite. The exact arrangement of the communication network however is immaterial for the invention.

The optical detection device—in the following referred to as “the camera”—can be arranged either as conventional digital camera or the digital camera in communication with a slit lamp, a pupillometer etc., suitable for acquisition of physiological features of the human eye. Physiological features of interest are the cornea, the retina, vessels within the retina and the papilla (the “blind spot”, where the optic nerve interfaces with the retina).

The medical imaging device 110 is a medical modality, for example an MRI or a CT.

The Processing unit 130 receives a digital image of a patient's eye, representing the physiological features as graphical/geometrical objects, referred to as “objects” in the following.

The acquisition of the digital image is arranged as a supplemental routine measure during routine medical check-ups such as diabetes or cancer screening programs.

The processing unit 130 may also receive an MRI image acquired from the medical imaging device 110.

The received digital image and/or the received MRI image are processed by the processing unit 130 to obtain a chronic disease risk index, in the following referred to as DRI. The processing of the digital image and/or the MRI image is based on reference data available on a reference database 150.

The processing unit 130 has appropriate interfaces for communication with the reference database 150 in order to acquire the reference data.

The DRI is indicative to a patient afflicted with a chronic disease such as diabetes, or cerebral and/or cardiovascular ailments or conditions.

Based on the DRI, appropriate treatment can be commenced or the patient can be scheduled for further diagnostic measures. The high reliability of the DRI and its wide scope for disease detection allows rendering further expensive diagnostic treatments superfluous. The inventive system therefore contributes to substantial savings to the health system.

The inventive method according to the present invention for calculating the DRI rests on the physiological observation that properties of certain physiological features within the human eye can be used advantageously for the detection of a large number of cerebral or cardiovascular chronic diseases.

The physiological features can be acquired in a comparably cheap manner by using the digital camera 120.

The tables 1, 2 and 3 show in a synoptical manner the physiological features (“locations”) and several of the properties (“eye defect”) along with the corresponding optical examination methods and the chronic diseases associated with the observed property.

The operation of the processing unit 130 will now be explained in more detail.

The processing unit 130 is either arranged as a software module on a storage medium or as a dedicated hardware chip.

The processing unit 130 comprises a number of dedicated tools for image processing and pattern recognition as known from packages such as XCALIPER for machine vision (MV) applications.

The processing unit 130 further comprises for the purposes of computing the DRI a suite of statistical tools. Such tools are commercially available for example in MATLAB™ from MATHWORKS®.

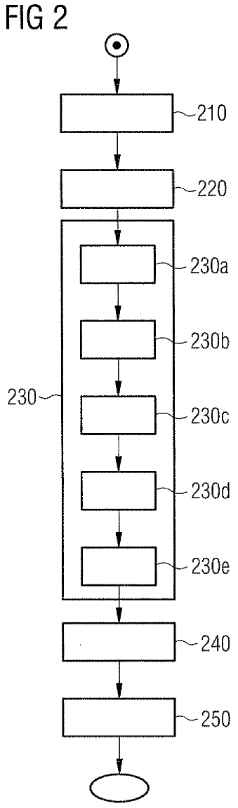

The interoperation of the image processing and statistical tools will now be explained with reference to FIG. 2.

In a first phase, previous to the processing of the digital image, reference values are acquired on the basis of previously acquired digital images.

The images of the papilla and the retina are subdivided into four sectors called superior nasal, superior temporal, inferior nasal and inferior temporal.

The reference values are to distinguish between healthy tissue and tissue afflicted with the chronic disease.

Properties of the images such as pixel intensity within the sectors are to be correlated with the skin colour of the person from whom images have been acquired. The correlation is necessary because the skin colour of the person has an impact on the properties of the objects properties. The reference values are then stored into the reference database 150 for later referral during actual processing step 230 during the processing phase.

The Processing phase commences with the acquisition of the digital image and/or the MRI image at steps 210 and steps 220, respectively.

The processing step 230 comprises a step 230a for extraction of the objects from the digital image representing the physiological features.

The step of extraction 230a further comprises a number of pre-processing steps in order to correct deficiencies in the digital image incurred during acquisition of the digital image. An image pre-processing tool for example uses a contrast to correct fuzziness in the digital image. Furthermore the image pre-processor uses filters to mitigate image noise in order to facilitate segmentation into regions and edges during a later segmentation step 230c to obtain the objects.

In this manner an enhanced digital image is obtained.

The pre-processing step requires no a prior knowledge about the objects.

Steps 230b and 230c effect statistical processing and segmenting/selecting the objects from the enhanced digital image. The steps 230b and 230c can be either combined into one step or can be effected separately.

Steps 230b and 230c are now explained in detail.

An image segmentation tool segments the enhanced digital image into a number of non overlapping regions and/or edges.

Some of the segments are later associated with specific ones of the objects, for example with cross-sections of veins and arteries within the retina object.

The segmentation tool uses decision functions—for example Bayes functions—incorporating some degree of medical knowledge about expected shapes, textures, contours and or pixel colours and or intensities or a weighted sum thereof. The knowledge is incorporated in form of parameters previously obtained from training samples.

Only those regions that are identified by the segmentation step as objects are further processed. The remaining regions are not further processed. The inventive method according to the present invention thus further achieves substantial data reduction.

In step 230b a gauging tool measures the properties of the objects into which the enhanced image has been segmented. Object properties are for example a size of the objects for example, area, girth widths and lateral and longitudinal lengths measured for example in pixels.

Spatial and other geometrical parameters are also gauged for example roundness and textures, both being based, for example, on spline-approximated curvatures of the objects.

Furthermore colour information is also gauged in terms of medium grey values or RGB values in case the digital image is a colour image or focal points in case the digital image is a binary image.

Furthermore, the spatial relationships between properties from different objects are measured. The ratio between the lateral width of the artery object and the vein object can be valuable clues for a cardiovascular condition. The lateral lengths can be measured in pixels or other suitable dimensional parameters.

Processing of further statistical parameters includes obtaining the sum of all veins diameters (SVD) and the sum of all arteries diameters (SAD), respectively.

Other statistical parameters are overall statistical parameters of the digital image. This comprises for example the average value of the image brightness, the variance of the average of the image contrast and corresponding higher moments, and entropy, both with respect to edges and textures as well as to pixel intensity. Again, the statistical processing of the overall statistical parameters is based, as in the first phase, on the subdivision of the digital image into the four sectors. The overall statistical parameters are acquired with respect to each of four sectors.

In step 230d and 230e the previously obtained reference values and rules in the database 150 are accessed.

Based on those rules and reference values the objects are classified in step 240 with respect to the acquired statistical parameters into healthy or not healthy with respect to a number of different diseases. Suitable statistical tests can be used for the classification, for example Student's T-test.

A percentage value is obtained, indicating a probability whether the patient is afflicted by a specific chronical disease. In this manner a vector of probabilities is obtained, the vector having one entry for each of the chronical diseases.

The MRI image is processed by the processing unit in a similar manner as the digital image explained above to obtain a vector of MRI statistical parameters.

In step 250 the statistical parameters can then be combined to calculate a combined vector of DRI values, for example by a weighted sum of all the corresponding entries of each of the two vectors.

A medical conclusion about the underlying disease or diseases can then be based on the DRI. The DRI and the two vectors of statistical parameters may also be stored to make them available for further medical evaluation.

The above description of illustrated embodiments of the invention is not intended to be exhaustive or to limit the invention to precise forms disclosed. While specific embodiments of, and examples for, the invention are described herein for illustrative purposes various equivalent modifications are possible within the scope of the invention and can be made without a deviating from the spirit and scope of the invention.

Further, the method might be implemented in software, in coded form. Alternatively, it is possible to implement the method according to the invention in hardware or hardware modules. The hardware modules are then adapted to perform the functionality of the steps of the method. Furthermore, it is possible to have a combination of hardware and software modules.

These and other modifications can be made to the invention with regard of the above detailed description. The terms used in the following claims should not be construed to limit the invention to the specific embodiments disclosed in the specification and the claims. Rather, the scope of the invention is to be determined entirely by the following claims, which are to be construed in accordance with established doctrines of claim interpretation.

Tables

TABLE 1
Orbita, Cornea and Pupil, examination methods and disease association

| | eye defect/failure | examination method | associated with |
|---|---|---|---|
| Orbita | Exophthalmus | Inspection | thyroid gland (Hyperthyreosis caused by Graves disease) |
| | (Endocrinopathy) | | |
| Cornea | Kayser-Fleischer Ring | Slit lamp: Brown-coloured stroma depositions | Wilson's disease (metabolic disorder) |
| | (Copper deposits | in peripheral, proximate limbal deep stroma | |
| | encircling the iris of the | of cornea | |
| | eye) | | |
| | Turbidity of peripheral | Slit lamp | Maroteaux-Lamy-Syndrome (metabolic disorder) |
| | cornea | | |
| Pupil | reflexive pupillary rigidity | Pupillometry: pupil diameter, Application of | Leutic diseases of central nervous system (Argyll-Robertson- |
| | | light: direct and indirect reaction; distance- | Syndrom), Encephalitis, multiple sclerosis |
| | | adaptation reaction | |
| | absolute pupillary rigidity | Pupillometry: pupil diameter, Application of | Malfunction in efferent duct, Edwinger-Westphal Nucleus, |
| | | light: direct and indirect reaction; distance- | Nervus oculomotorius, Iris musculature |
| | | adaptation reaction | |
| | Mydriasis paralytica | Pupillometry: pupil diameter, Application of | single-sided: oculomotoriusparesis = absolute pupillary rigidity; |
| | | light: direct and indirect reaction; distance- | double-sided: Atropin-intoxication, spasmolytica, Anti-Parkinson |
| | | adaptation reaction | medicaments, Antidpressants, Botulism and Carbon monoxide |
| | Miosis spastica | Pupillometry: pupil diameter, Application of | single-sided: subdural hematoma; double-sided: Morphium |
| | | light: direct and indirect reaction; distance- | abuse, deep narcosis, mushroom intoxication, states of cerebral |
| | | adaptation reaction | irritation, encephalitis, meningitis and reflexive pupillary rigidity |
| | Miosis paralytica | Pupillometry: pupil diameter; Application of | almost everytime single-sided, paralysis of sympathetic nervous |
| | | light: direct and indirect reaction; distance- | system (combined with Ptosis and Enophthalmus: Horner |
| | | adaptation reaction | syndrome) |
| | Mydriasis spastica | Pupillometry: pupil diameter, Application of | single-sided: occurs with pulmonic, cardiac und abdominal |
| | | light: direct and indirect reaction; distance- | processes (local irritation of sympathetic nervous system, |
| | | adaptation reaction | stellate ganglion); double-sided: Migraine, Schizophrenia, |
| | | | Hyperthyreosis, cocaine intoxication, as well as with hysterical |
| | | | and epileptical attacks |

TABLE 2
Papilla and optic nerve, examination methods and disease association

| | eye defect/failure | examination method | associated with |
|---|---|---|---|
| Papilla | Micropapilla | Ophthalmoscopy: small-sized Papilla (<<2.7 mm³) | Mikrocephaly/Coloboma/ |
| and | | | Microophthalmus |
| optic | papilla turgidity | | systemic disease > increased |
| nerve | | | pressure in optic nerve |
| | | | sheath and venostasis |
| | papilledema | Ophthalmoscopy: Papilla edematous, hyperaemic and lacking defined | Increase of intracranial pressure |
| | | borders, radiary, stripe-shaped bleedings at edge of papilla; Perimetry: | caused by expansive |
| | | increased size of blind spot | cerebral processes, |
| | | | e.g. tumour, brain abscess, |
| | | | meningitis, encephalitis, traumatic |
| | | | brain injury, cerebral bleedings, . . . |
| | Neuritis retrobulbaris | Perimetry: central scotoma; high-grade reduction of visual acuity; visual | amongst others: early symptoma |
| | | evoked cortical potential; delay of impulse processing in Nervus opticus | of Multiple Sclerosis |
| | Anterior ischaemic | very high-grade reduction of visual acuity; Ophthalmoscopy: Papilla | Arteriosclerosis, often with diabetics |
| | neuropathy | edematous, little prominent, pale and showing miniscule bleedings; | (diabetic papillopathy); |
| | of Nervus Opticus | Perimetry: defective field of view; pupil reaction; A maurotic pupillary | embolic vascular |
| | | rigidity | obliteration, e.g. with atrial |
| | | | fibrillation, endocarditis, . . . |
| | Arteritis temporalis (Temporal | very high-grade reduction of visual acuity; Ophthalmoscopy; Papilla | granulomatous vasculitis |
| | arteritis) | edematous, little prominent, pale and showing miniscule bleedings; | |
| | Atrophy of Nervus Opticus | | |
| | Toxic Atrophy of Nervus | Perimetry: central scotoma; Ophthalmoscopy: pale papilla, visible | abuse of alcohol and/or tabac, |
| | Opticus | Lamina cribrosa; no progressing after discontinuation of noxa | intoxication caused by Methanol, |
| | | | lead, arsenic, Thallium, |
| | | | Chinin or Ethambutol |
| | Hereditary Atrophy of Nervus | Perimetry: central scotoma; Ophthalmoscopy: pale papilla, visible | Association mit Multiple |
| | Opticus (Liver-Opticus- | Lamina cribrosa | Sclerosis (genetic correlation) ? |
| | Atrophy) | | |
| | Ascending Atrophy of Nervus | Ophthalmoscopy: Papilla yellowish pale and slightly diffuse borders | damage of ganglion cell layers and |
| | Opticus | | nerve fiber layers caused by |
| | | | Chorioretinits or |
| | | | central artery occulsion |
| | Descending Atrophy of | Ophthalmoscopy: Papilla pale, diffuse borders | Hydrocephalus internus, |
| | Nervus Opticus | | tumour compression |
| | Glaucomatous Atrophy of | Ophthalmoskopie: Excavation of Papilla by ½ of Papilla diameter, | cardiovascular diseases, Diabetes |
| | Nervus Opticus | progredient atrophy, down-bending of vessels, glaucomatous ring | |

TABLE 3
Visual pathway, examination method and disease association

| | eye defect/failure | examination method | associated with |
|---|---|---|---|
| Visual | Chiasma syndrome | single- or double-sided reduction of visual acuity; | Pituitary gland adenoma, |
| Pathway | | Perimetry: heteronymous, bitemporal Hemianopsia (half- | Meningioma, Craniopharyngioma, . . . |
| (Symptoms: | | sided defective field of view); Ophthalmoscopy: | |
| Failures in field | | descending Atrophy of Nervus Opticus: MRI | |
| of view and | Lesions of Tractus opticus | Perimetry: Homonymous defects of visual acuity; | Infarctions, tumours, bleedings or |
| atrophic Nervus | and Corpus geniculatum | Ophthalmoscopy: possibly moderate Atrophy of Nervus | demyelinizing diseases in the |
| Opticus) | laterate | Opticus; MRI | area of temporal lobe, |
| | | | Mesencephalon, Thalamus |
| | | | and Internal Capsule |
| | Lesions of Optic Radiation | Perimetry: Homonymous defects of visual acuity or | Infarctions, tumours, bleedings or |
| | | Quadrant anopias, but no Atrophy of Nervus Opticus; MRI | softening spots in the area of |
| | | | Internal Capsule, temporal/parietal/ |
| | | | occipital lobe |
| | lesions of visual cortex | Perimetry: Homonomous defects of visual acuity or | Infractions, tumours, bleedings, |
| | | Quadrantanopias; MRI | vessel spasms, softening spots or tumours |
| | | | occipital brain |

What we claim is:

1. A computer-implemented method for calculating a chronic disease risk index for a patient undergoing a first examination, the method comprising:
providing a first result of the first examination, wherein the first examination is selected from a group of medical imaging methods;
providing a second result of a second examination, the second examination being related to an examination of the patient's eye;
processing the first result in combination with the second result such as to calculate a combined result, wherein the combined result relates to both the first and the second result;
classifying the combined result;
calculating the risk index for the patient, the risk index being based on the combined result or on the classified common result.

2. Computer-implemented method according to claim 1, wherein providing the first result or providing the second result comprise:
carrying out an additional second examination concomitantly to the first examination.

3. Computer-implemented method according to claim 1, wherein the first examination and the second examination are performed in combination or are performed separately.

4. Computer-implemented method according to claim 1, wherein the first result or the second result comprise at least:
detecting the first result and the second result of at least a previous examination;
matching those results from the group of the first result and the second result which have the same detection times.

5. Computer-implemented method according to claim 1, wherein processing of the first result or processing the second result comprise at least:
extracting relevant features from the first result or from the second result;
statistically processing the first result or the second result;
selecting relevant parts of the first result or the second result or segmenting an image in relevant image segments;
accessing a database for using rules, the rules being adapted to relate the first result to the second result in order to form a combined result;
accessing a database for comparing the first result or the second result with reference values for evaluating a conclusion with respect to the chronic disease risk index for the patient.

6. Computer-implemented method according to claim 1, wherein the method is based on predefinable patterns wherein the first result or the second result is compared with the predefinable patterns or properties of reference objects.

7. Computer-implemented method according to claim 1, wherein the method is based on automatic image recognition.

8. Computer-implemented method according to claim 1, wherein the method is performed automatically.

9. Computer-implemented method according to claim 1, wherein the method comprises at least one of the following:
storing an intermediate result of the method;
storing the chronic disease risk index as a final result;
storing a conclusion, wherein the conclusion is based on the chronic disease risk index.

10. Computer-implemented method according to claim 1, wherein the second examination is related to the examination of the patient's eye, particularly to an imaging examination of several parameters of the eye, particularly imaging examinations of an optical disc, at least one of a vessel and a cornea and retina of the patient's eye.

11. Computer-based device for calculation of a chronic disease risk index for a patient undergoing a first examination, the device comprising:
at least an imaging device, which is adapted to perform a first examination and to provide a corresponding first result;
an optical detection device, which is adapted to perform a second examination with respect to the patient's eye and to provide a second corresponding result;
wherein the device is adapted to communicate with a separate processing unit or which is adapted to comprise at least a processing unit, wherein the processing unit is adapted to process the first result and the second result in combination such as to calculate a combined result, and to classify the combined result and wherein the processing unit is further adapted to calculate the chronic disease risk index for the patient, the disease risk index being based on the common result.

12. Computer-based system for calculation of a chronic disease risk index for a patient, undergoing a first examination, the system comprising:
at least an imaging device, which is adapted to perform a first examination and to provide a corresponding first result;
an optical detection device, which is adapted to perform a second examination with respect to the patient's eye and to provide a second corresponding result;
a processing unit which is adapted to process the first result and the second result in combination such as calculate a combined result, to classify the common result and which is further adapted to calculate the chronic disease risk index for the patient being based on the common result;
Interfaces between the imaging device, the optical detection device and the processing unit.

13. Computer-based system according to claim 12, wherein the optical detection device comprises at least one of a slit-lamp and a digital camera, pupillometer, optical coherence tomographic device, ophthalmoscope, fluorescence or angiographic device.

14. A computer readable medium having computer-executable instructions for performing a method according to claim 1.

15. A computer product having computer-executable instructions for performing a method according to claim 1.
